(19) United States
(12) Patent Application Publication
(10) Pub. No.: US 2016/0008506 A1
(43) Pub. Date: Jan. 14, 2016

(54) STEM CELL COMPOSITIONS AND METHODS FOR WOULD HEALING

(71) Applicant: NEOSTEM, INC., New York, NY (US)

(72) Inventor: Vincent FALANGA, Boston, MA (US)

(21) Appl. No.: 14/773,099

(22) PCT Filed: Mar. 7, 2014
(86) PCT No.: PCT/US2014/022159

§ 371 Date: Sep. 4, 2015

Related U.S. Application Data
(60) Provisional application No. 61/774,489, filed on Mar. 7, 2013.

(51) Int. Cl. A61L 26/00; A61K 38/48; A61K 38/36; A61K 35/545

(52) U.S. Cl. CPC A61L 26/0057; A61L 26/0076; A61L 26/0052; A61L 26/0004; A61K 35/545; A61K 38/4833; A61K 38/363; A61L 2430/34; A61L 2300/64; A61L 2300/418; C12Y 304/21005

(57) ABSTRACT

This application provides compositions for wound repair comprising VSEL stem cells and methods for treating acute and chronic cutaneous wounds, including burns and ulcers.

### CROSS-REFERENCE TO RELATED APPLICATIONS

This application claims priority to U.S. Application No. 61/774,489, filed Mar. 7, 2013, which is incorporated herein by reference in its entirety.

### FIELD OF THE INVENTION

This application relates to compositions and methods of preparing and using VSEL stem cells for wound healing applications.

### BACKGROUND OF THE INVENTION

Chronic wounds remain a formidable challenge. In spite of recent advances from breakthroughs in recombinant growth factors and bioengineered skin, up to 50% of chronic wounds that have been present for more than a year remain resistant to treatment.

Fibrin sealants are a type of surgical “glue” that is made from human blood-clotting proteins, and are typically used during surgery to control bleeding. Fibrin sealants have been used to augment hemostasis, seal tissues, facilitate targeted delivery of drugs, and in treatments of wounds. Examples of such sealants are found in U.S. Patent Publication Nos. 2008/0181879, 2008/0199513, 2004/0229333, and 2006/0240555, incorporated herein by reference in their entireties. Certain of the fibrin sealant compositions comprise stem cells. See e.g., U.S. Patent Publication No. 2011/0212062. While there have been advances in the treatment of difficult-to-heal wounds in the last few years, there is still a considerable number of acute and chronic wounds that may be extensive and require treatment of large areas and/or large numbers of stem cells. Thus, there remains a need to efficiently stimulate the healing of acute and chronic wounds.

### SUMMARY OF THE INVENTION

The present invention provides compositions of, and methods for using, a fibrin sealant comprising VSEL stem cells and delivery systems for same. The sealants of the invention provide a matrix for contacting stem cells with a wound, but need not have the strength or structural integrity of fibrin compositions used to close or cover wounds, or protect wounds from the environment. According to some embodiments, the invention provides a method for topically applying a fibrin sealant to a site of a wound, wherein the fibrin sealant is produced by admixture of a fibrinogen complex component, a thrombin component, and a VSEL stem cell component. The cellular component may comprise an admixture of VSEL stem cells and other cells. The inventor has discovered that surprisingly small numbers of VSELs can be administered, yet provide effective wound healing. According to preferred embodiments, the fibrin sealant comprises an amount of VSELs that provides an amount at the wound of 10⁵ VSEL stem cells or fewer per cm² of wound area. As used herein, amounts or doses of VSELs per cm² refer to the surface area of the wound (i.e., not the surface area of the subject being treated). The discovery significantly reduces the amount of stem cells needed for treatment and allows for more extensive treatment from a given amount of stem cells.

According to some embodiments, the fibrin sealant is produced by combining a fibrinogen complex (FC) component, thrombin component, and a cellular component in admixture. The FC/thrombin ratio may be between the range of 2 to 10 mg/ml fibrinogen per 25 U/ml thrombin. The FC/thrombin ratio is preferably in the range of about 5 mg/ml fibrinogen per 25 U/ml thrombin.

According to some embodiments, the fibrin sealant is produced by combining a fibrinogen complex (FC) component and a thrombin component in admixture. In another embodiment, the cellular component may be added to the FC component before admixture of the FC component with the thrombin component. According to some embodiments, the cellular component may be added to the thrombin component. According to some embodiments, cellular component may be added to the mixture of FC and thrombin before the components are allowed to form the fibrin gel.

According to preferred embodiments, protease inhibitors are excluded from the fibrin sealant compositions of the present invention. While fibrin sealants may often contain a protease inhibitor to extend the life of the sealant once applied, in the instant invention, once a stem cell-containing fibrin sealant has been applied to a would, it may be preferable not to inhibit degradation of the fibrin sealant for an extended period of time.

The invention also provides methods for impregnating or seeding a fibrin sealant to form a therapeutic formulation for the treatment of acute and chronic wounds. Such methods include administering to a patient a fibrin sealant comprising a cellular component. According to some embodiments, the fibrin sealant is in the form of a polymerized gel or spray. The fibrin sealant of the present invention may be applied one or more to the site of the wound one or more times as needed during the course of the healing process, such as once, twice, three times, four times, five times, or more.

According to some embodiments, the invention provides for methods of treating acute or chronic wounds. Generally, the patient is suffering from a wound which would benefit from the compositions and methods described herein, which would be apparent to one of ordinary skill in the art. According to preferred embodiments, the wound is a chronic cutaneous wound.

In one embodiment, the fibrin sealant is administered to a patient using methods well-known in the art, such as injection, spray, endoscopic administration or pre-formed gel and other methods known to one of ordinary skill in the art.

The present invention provides a method of using a wound sealant, comprising: a) combining VSEL stem cells with a fibrin sealant to form a wound sealant, said matrix comprising calcic thrombin and fibrinogen, wherein the concentration of calcic thrombin is about 25 U/ml, wherein the concentration of fibrinogen is from about 2 to about 5 mg/ml, and wherein the final concentration of VSEL stem cells provides a dosage of 10⁵ or fewer VSEL stem cells/cm²; and b) administering the wound sealant to a cutaneous wound, wherein the wound sealant is administered in the form of a polymerized gel or spray. In certain embodiments, the dosage of VSELs per cm² is on the order of 10², 10³, or 10⁴ cells.

According to preferred embodiments, the present invention provides a method of ameliorating the formation of scars at a wound site, comprising: a) combining VSEL stem cells with a fibrin sealant to form a wound sealant, said fibrin sealant comprising calcic thrombin and fibrinogen, wherein the concentration of calcic thrombin is about 25 U/ml, wherein the concentration of fibrinogen is from about 2 to about 5 mg/ml, and wherein the final concentration of VSEL stem cells is about 10⁵/cm² or fewer; b) administering the wound sealant to a wound site, wherein the wound sealant is administered in the form of a polymerized gel or spray.

According to preferred embodiments, the present invention provides a method of treating scleroderma comprising: a) combining VSEL stem cells with a fibrin sealant to form a wound sealant, said fibrin sealant comprising calcic thrombin and fibrinogen, wherein the concentration of calcic thrombin is about 25 U/ml, wherein the concentration of fibrinogen is from about 2 to about 5 mg/ml, and wherein the final concentration of VSEL stem cells is about 10⁵/cm²or fewer; b) administering the wound sealant to a scleroderma ulcer, wherein the wound sealant is administered in the form of a polymerized gel or spray.

According to preferred embodiments, the present invention provides a method of making a wound sealant comprising: providing a fibrinogen complex (FC) component, a calcic thrombin component, and a cellular component; adding the cellular component to the FC component before admixture of the FC component with the calcic thrombin component; and adding the calcic thrombin component to the combined FC/cellular component mixture, wherein the concentration of fibrinogen is from about 2 to about 5 mg/ml, and wherein the final concentration of VSEL stem cells provides an application of about 10⁵ or fewer VSEL stem cells/cm² of wound area. The concentration of VSELs in the wound repair composition is determined from the area to be treated, the number of cells to be applied per unit area, and the amount of the composition that will cover the area. For example, 100 microlitres of wound repair sealant was used to treat certain exemplary mouse tail wounds, and the requisite number of cells was dispersed in that volume. According to the invention, the VSEL stem cells can be freshly isolated, or previously aliquoted, stored, and/or cyropreserved.

In certain embodiments, the fibrin sealant is administered to the site of the wound at least two times over the span of three weeks. According to preferred embodiments, the fibrin sealant is administered to the site of the wound in the form of a spray at a CO₂ psi of less than 5 psi. According to preferred embodiments, the fibrin sealant is topically applied to the site of the wound. According to preferred embodiments, a skin substitute is applied to the site of the wound.

The invention also provides a kit for preparing a fibrin sealant comprising, a) a first vial or first storage container containing a fibrinogen complex component, wherein the vial optionally comprises a cellular component, and b) a second vial or second storage container having a thrombin component, said kit optionally containing a third vial or third storage container having a cellular component when said first vial or first storage container does not include a cellular component, said kit further containing instructions for use thereof. The kit may also comprise instruments for use or administration of the fibrin sealant in vitro or in vivo. The kit may also comprise a means for characterizing the cellular component. Such means includes reagents for identifying the presence of stem/progenitor cellular markers. Reagents for identifying the presence of stem/progenitor cellular marker include, but are not limited to, antibodies.

The invention also provides a kit for preparing a fibrin sealant comprising, a) a first vial or first storage container containing a fibrinogen complex component, and b) a second vial or second storage container having a thrombin component, wherein the vial optionally comprises a cellular component, said kit optionally containing a third vial or third storage container having a cellular component when said second vial or second storage container does not include a cellular component, said kit further containing instructions for use thereof. The kit may also comprise instruments for use or administration of the fibrin sealant in vitro or in vivo. The kit may also comprise a means for characterizing the cellular component.

The invention also provides a kit for preparing a fibrin sealant comprising, a) a first vial or first storage container containing a fibrinogen complex component, wherein the concentration of fibrinogen is from about 2 to about 5 mg/ml, and b) a second vial or second storage container having a thrombin component, said kit further containing instructions for use thereof. According to some embodiments, the kits of the present invention provide that the concentration of thrombin is about 25 U/ml. According to some embodiments, the kits of the present invention provide that the first vial or first storage container optionally comprises a cellular component. According to some embodiments, the kits of the present invention provide that the optional third vial or third storage container having a cellular component when said first vial or first storage container does not include a cellular component. According to some embodiments, the kits of the present invention provide may further comprise instruments for use or administration of the fibrin sealant in vitro or in vivo.

According to some embodiments, the kits of the present invention provide may further comprise reagents for characterizing the cellular component.

Other features and advantages of the invention will become apparent from the following detailed description. It should be understood, however, that the detailed description and the specific examples, while indicating specific embodiments of the invention, are given by way of illustration only, because various changes and modifications within the spirit and scope of the invention will become apparent to those skilled in the art from this detailed description.

### BRIEF DESCRIPTION OF FIGURES

FIG. 1. Left Panel: Reepithelialization observed in response to treatment with VSELs compared to fibrin control after 7 days. Right Panel: % reepithelialization 7 and 14 days post wounding and treatment comparing 2500 human VSELs, 500,000 human MSCs, and fibrin control.

FIG. 2. Histology and effect on healing of a representative mouse tail wound 7 days after application of human VSEL stem cells in a fibrin spray. Biopsy specimens were stained with hematoxylin and eosin (H&E). The fibrin spray contained VSEL stem cells in an amount necessary to apply the indicated numbers of VSEL stem cells to the wounds. (A) Representative effect of 2,500 or 400 VSELs (similar histology was observed for both doses). The solid arrows represent the initial sites of wounding. Notice skin appendages (e.g., hair follicles, undulating surface of epithelium) are left behind. The dashed arrows are the limits to which the epidermis has migrated at Day 7, almost completing closure of the wound. (B) Day 7 following application of fibrin spray alone. The solid arrows represent the initial sites of wounding. The dashed arrows show poor migration from the original site of injury (solid arrows). The upper side, above the space, is crust and bacteria, which are expected. (C) Wound re-epithelialization assessed by % migration of the epidermis from the initial site of injury. The bars are standard deviation (SD). The doses of 2,500 and 400 VSELs were equivalent, and both superior to the control (with a p value at least <0.05 in terms of statistical significance.

### DETAILED DESCRIPTION

The invention provides compositions that promote wound healing, which comprise VSELs. The VSELs are typically incorporated into a matrix, such as a matrix formed from fibrinogen, referred to herein as a sealant. Thus, the wound sealants of the invention comprise a matrix component and a cellular component. Generally, the cellular component promotes or assists wound healing, while the matrix component provides support functions for the cellular component, including, but not limited to one or more of maintaining the cellular component in proximity with the wound, maintaining a biological function of the cellular component, and/or providing a protective barrier.

The wound sealants of the invention feature a therapeutically effective amount of VSEL stem cells. Advantageously, it has been discovered that surprisingly small numbers of VSEL stem cells are effective to promote wound healing.

The term “very small embryonic-like stem cell” is also referred to herein as “VSEL stem cell” or “VSEL.” The term has been used to refer to stem cells obtained from adult tissue (e.g., without limitation, bone marrow, blood, cord blood) that can differentiate into certain cell types of all three germ layers. In certain embodiments, the VSEL stem cells (“VSELs”) are human VSELs and may be characterized as lin⁻, CD45⁻, and CD34⁺. In certain embodiments, the VSELs are human VSELs and may be characterized as lin⁻, CD45⁻, and CD133⁺. In certain embodiments, the VSELs are human VSELs and may be characterized as lin⁻, CD45⁻, and CXCR4⁺. In some such embodiments, the VSELs are lin⁻, CD45⁻, CXCR4⁺, and CD34⁺. In other such embodiments, the VSELs are lin⁻, CD45⁻, CXCR4⁺, and CD34⁻. In certain embodiments, the VSELs are human VSELs and may be characterized as lin⁻, CD45⁻, CXCR4⁺, CD133 and CD34⁺. In the above embodiments, human VSELs may further express at least one of SSEA-4, Oct-4, Rex-1, and Nanog. VSELs may also be characterized as possessing large nuclei surrounded by a narrow rim of cytoplasm, and containing embryonic-type unorganized chromatin. In some embodiments, VSELs have high telomerase activity. In certain embodiments, human VSELs may be characterized as lin⁻, CD45⁻, CXCR4⁺, CD133 Oct 4⁺, SSEA4⁺, and CD34⁺. In certain embodiments, the human VSELs may be less primitive and may be characterized as lin⁻, CD45⁻, CXCR4⁺, CD133⁻, and CD34⁺. In certain embodiments, the human VSELs may be enriched for pluripotent embryonic transcription factors, e.g., Oct-4, Sox2, and Nanog. In certain embodiments, the human VSELs may have a diameter of 4-5 μm, 4-6 μm, 4-7 μm, 5-6 μm, 5-8 μm, 6-9 μm, or 7-10 μm. VSELs administered according to the invention can be collected and enriched or purified and used directly, or frozen for later use. VSELs from cord blood have also been characterized as being CD133⁺/GlyA⁻/CD45⁻. In some embodiments, the CD133⁺/GlyA⁻/CD45⁻ cells are ALDH^{high} cells. In some embodiments, the CD133⁺/GlyA⁻/CD45⁻ cells are ALDH^{low} cells. (See, e.g., WO 2010/057110, entitled Methods And Compositions For Long Term Hematopoietic Repopulation).

VSEL stem cells are collected from an autologous, allogeneic, or heterologous human or animal source. An autologous animal or human source is usually preferred, but as described further herein, allogeneic VSEL stem cells can also be employed in treatments. Stem cell-containing sealants are then prepared and isolated as described herein. As mentioned, surprisingly few VSELs are effective to promote wound healing. Thus, sealants of the present invention comprise a cellular component comprising an amount of VSEL stem cells to provide a sealant dosage of about 10⁵ VSEL stem cells/cm² or fewer. According to some embodiments, the dosage of VSEL stem cells is about 10⁴ VSEL stem cells/cm² or fewer. According to some embodiments, the dosage of VSEL stem cells is about 10³ VSEL stem cells/cm² or fewer. According to some embodiments, the dosage of VSEL stem cells is about 2.5×10² VSEL stem cells/cm² or fewer. According to some embodiments, the dosage of VSEL stem cells is about 10² VSEL stem cells/cm² or fewer.

In certain embodiments, the dosage of VSEL stem cells is from about 10² to 10³ VSEL stem cells/cm². In certain embodiments, the dose of VSEL stem cells is from about 5×10² to 5×10³ VSEL stem cells/cm². In certain embodiments, the dosage of VSEL stem cells is from about 10³ to 10⁴ VSEL stem cells/cm². In certain embodiments, the dose of VSEL stem cells is from about 5×10³ to 5×10⁴ VSEL stem cells/cm². In certain embodiments, the dose of VSEL stem cells is from about 10⁴ to 10⁵ VSEL stem cells/cm².

The cellular component comprises purified VSELs. As used herein, a cell exists in a “purified form” when it has been isolated away from all other cells that exist in its native environment, but also when the proportion of that cell in a mixture of cells is greater than would be found in its native environment. Stated another way, a cell is considered to be in “purified form” when the population of cells in question represents an enriched population of the cell of interest, even if other cells and cell types are also present in the enriched population. A cell can be considered in purified form when it comprises in some embodiments at least about 10% of a mixed population of cells, in some embodiments at least about 20% of a mixed population of cells, in some embodiments at least about 25% of a mixed population of cells, in some embodiments at least about 30% of a mixed population of cells, in some embodiments at least about 40% of a mixed population of cells, in some embodiments at least about 50% of a mixed population of cells, in some embodiments at least about 60% of a mixed population of cells, in some embodiments at least about 70% of a mixed population of cells, in some embodiments at least about 75% of a mixed population of cells, in some embodiments at least about 80% of a mixed population of cells, in some embodiments at least about 90% of a mixed population of cells, in some embodiments at least about 95% of a mixed population of cells, and in some embodiments about 100% of a mixed population of cells, with the proviso that the cell comprises a greater percentage of the total cell population in the “purified” population that it did in the population prior to the purification. In this respect, the terms “purified” and “enriched” can be considered synonymous.

Accordingly, the sealants of the present invention comprise VSEL stem cells and may include amounts of other cell types, including but not limited to adult bone marrow-derived stem cells, adult PBSCs, or adult MSCs.

According to the invention, VSELs are preferably delivered in a preparation that maintains the VSELs in proximity with a wound or wound bed and maintains the viability of the VSELs. In one embodiment, the preparation comprises a matrix component provides support for the cellular component, including, but not limited to one or more of maintaining the cellular component in proximity with the wound, maintaining one or more biological functions of the cellular component, and/or providing a protective barrier. In certain embodiments, the VSEL-containing compositions of the invention can be in the form of a void-filling composition, or a composition disposed between tissue layers. For example, a VSEL-containing composition of the invention may be applied in contact with a wound bed and a tissue graft.

In certain embodiments, the matrix component is referred to herein as a “sealant.” The term sealant highlights that the composition, once applied, is retained at the site of application. The term does not imply that a film, impervious to migration or diffusion, for example of cells, growth factors, drugs, or other agents, is formed. In this regard, in certain embodiments, the matrices and sealants of the invention, once administered at the site of a wound, may be separately covered or coated to protect the wound site.

In certain embodiments, the cell matrix allows for cellular migration. For example, cells provided in the cellular component of the matrix may enter the wound to affect a therapeutic response. In certain embodiments, VSELs assist wound healing by promoting migration of epidermal cells and fibroblasts of the subject being treated into the wound.

As exemplified, the matrix component comprises two human plasma-derived components: (a) a purified and concentrated Fibrinogen Complex (FC) composed primarily of fibrinogen and fibronectin along with catalytic amounts of Factor XIII and plasminogen and (b) a high potency thrombin. Such fibrin sealants may also contain aprotinin. By the action of thrombin, (soluble) fibrinogen is at first converted into fibrin monomers which aggregate spontaneously and form a so-called fibrin clot. Simultaneously, factor XIII (FXIII) present in the solution is activated by thrombin in the presence of calcium ions to factor XIIIa. The aggregated fibrin monomers and any remaining fibronectin possibly present are cross-linked to form a high molecular weight polymer by new peptide bonds forming. By this cross-linking reaction, the strength of the clot formed is substantially increased. See e.g., U.S. Patent Publication No. 2008/0181879, incorporated herein by reference in its entirety. Generally, the clot adheres well to wound and tissue surfaces, which leads to the adhesive and hemostatic effect. Therefore, fibrin adhesives are frequently used as two-component adhesives which comprise a fibrinogen complex (FC) component together with a thrombin component which additionally contains calcium ions. One such commercially available fibrin sealant is TISSEEL (Baxter). TISSEEL consists of a two-component fibrin biomatrix that offers highly concentrated human fibrinogen to seal tissue and stop diffuse bleeding. Other commercially available fibrin sealants include BERIPLAST® (Behringwerke AG, Marburg/Lahn, FRG) and BIOCOL (CRTS, Lille, France).

The fibrin sealants exemplified in the present invention are produced by combining a fibrinogen complex (FC) component, a thrombin component, and further a cellular component. According to some embodiments, the cellular component may be added to the FC component before the addition of the thrombin component. According to some embodiments, the cellular component may be added to the thrombin component. According to some embodiments, cellular component may be added to the mixture of FC and thrombin before the components are allowed to form the fibrin gel.

It has been found advantageous to limit the degree to which the VSEL component is restrained in the sealant matrix and to allow or promote migration of host cells, such as epidermal cells, during wound reepithelialization. Accordingly, the invention provides ranges for the concentration and ratios of the matrix components. The FC/thrombin ratio may be between the range of 2 to 10 mg/ml fibrinogen per 25 U/ml thrombin, which includes, for example, about 2 mg/ml fibrinogen per about 25 U/ml thrombin, about 2.5 mg/ml fibrinogen per about 25 U/ml thrombin, about 3 mg/ml fibrinogen per about 25 U/ml thrombin, about 3.5 mg/ml fibrinogen per about 25 U/ml thrombin, about 4 mg/ml fibrinogen per about 25 U/ml thrombin, about 4.5 mg/ml fibrinogen per about 25 U/ml thrombin, about 5 mg/ml fibrinogen per about 25 U/ml thrombin, about 5.5 mg/ml fibrinogen per about 25 U/ml thrombin, about 6 mg/ml fibrinogen per about 25 U/ml thrombin, about 6.5 mg/ml fibrinogen per about 25 U/ml thrombin, about 7 mg/ml fibrinogen per about 25 U/ml thrombin, about 7.5 mg/ml fibrinogen per about 25 U/ml thrombin, about 8 mg/ml fibrinogen per about 25 U/ml thrombin, about 8.5 mg/ml fibrinogen per about 25 U/ml thrombin, about 9 mg/ml fibrinogen per about 25 U/ml thrombin, about 9.5 mg/ml fibrinogen per about 25 U/ml thrombin, and about 10 mg/ml fibrinogen per about 25 U/ml thrombin. The FC/thrombin ratio is preferably in the range of about 5 mg/ml fibrinogen per about 25 U/ml thrombin. Preferred ranges for the FC/thrombin ratio include between about 4 to about 6 mg/ml fibrinogen per about 25 U/ml thrombin, between about 4.5 to about 6 mg/ml fibrinogen per about 25 U/ml thrombin, between about 5 to about 6 mg/ml fibrinogen per about 25 U/ml thrombin, between about 5.5 to about 6 mg/ml fibrinogen per about 25 U/ml thrombin, between about 4 to about 5.5 mg/ml fibrinogen per about 25 U/ml thrombin, between about 4 to about 5 mg/ml fibrinogen per about 25 U/ml thrombin, and between about 4 to about 4.5 mg/ml fibrinogen per about 25 U/ml thrombin.

According to preferred embodiment, the fibrinogen density/concentration is between the range of about 2 to about 10 mg/ml, which includes, for example, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, and about 10 mg/ml fibrinogen. The fibrinogen density is preferably between the range of about 2 to about 5 mg/ml. Preferred ranges for the fibrinogen density/concentration include between about 4 to about 6 mg/ml, between about 4.5 to about 6 mg/ml, between about 5 to about 6 mg/ml, between about 5.5 to about 6 mg/ml, between about 4 to about 5.5 mg/ml, between about 4 to about 5 mg/ml, between about 4 to about 4.5 mg/ml, between about 2 to about 6 mg/ml, between about 2 to about 5 mg/ml, between about 2.5 to about 5 mg/ml, between about 3 to about 5 mg/ml, and between about 3.5 to about 5 mg/ml. Thrombin is added to the fibrinogen in an amount sufficient to form a polymerized gel within between about 3 seconds to about 120 seconds. Preferably, between about 3 seconds to about 60 seconds, between about 3 seconds to about 30 seconds, between about 3 seconds to about 20 seconds, between about 3 seconds to about 10 seconds, between about 10 seconds to about 60 seconds, between about 20 seconds to about 60 seconds, between about 30 seconds to about 60 seconds, and between about 30 seconds to about 120 seconds.

According to some embodiments, the foundation for the wound sealants of the present invention may be any protein or any non-protein polymer (e.g., CAVILON™ from 3M) that is capable of forming a matrix and capable of supporting or encapsulating the cells or cellular component. The density/concentration of the polymer may be between 2.5 to 10 mg/ml. The polymer density/concentration may be between the range of about 2 to about 10 mg/ml, which includes, for example, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, and about 10 mg/ml. The polymer density is preferably in the range of about 2 to about 5 mg/ml. Preferred ranges for the polymer density include between about 4 to about 6 mg/ml, between about 4.5 to about 6 mg/ml, between about 5 to about 6 mg/ml, between about 5.5 to about 6 mg/ml, between about 4 to about 5.5 mg/ml, between about 4 to about 5 mg/ml, between about 4 to about 4.5 mg/ml, between about 2 to about 6 mg/ml, between about 2 to about 5 mg/ml, between about 2.5 to about 5 mg/ml, between about 3 to about 5 mg/ml, and between about 3.5 to about 5 mg/ml.

According to preferred embodiments, the fibrin sealants are in the form of a polymerized gel or gel spray. The concentration of each of the components are preferably optimized to provide a gel that allows for migration of applied stem cells and cells of the tissue being treated. Preferably, when provided in spray form, gel formation occurs upon or soon after application (e.g., to prevent running after application). Optionally, cell-containing fibrin sealant gel may be covered for protection after administration.

Once administered to a wound, the stem cell-containing sealant should be kept in position for a sufficient period of time, for example to allow for migration of cells from the sealant into the wound bed. The period of time can be, e.g., at least one hour, at least two hours, at least three hours, at least four hours, one to two hours, two to four hours, four to six hours, six to eight hours, eight to 12 hours, 12 to 16 hours, or 16 to 24 hours. The stem cell-containing sealant can be kept in position using, for example, a wound dressing that does not absorb or disrupt the sealant. In one exemplary embodiment, a spray on polymer such as Cavilon (3M) is sprayed over the gel sealant.

The compositions and methods of the invention disclosed herein are useful for treating a patient having acute or chronic wounds. Acute wounds include, without limitation, burns and abrasions and wounds resulting from skin removal e.g., for grafting, or to remove a cancerous lesion. Chronic wounds include, but are not limited to the following: chronic ischemic skin lesions; scleroderma ulcers; arterial ulcers; diabetic foot ulcers; pressure ulcers; venous ulcers; nonhealing lower extremity wounds; ulcers due to inflammatory conditions, and/or long-standing wounds. Although particular embodiments are exemplified herein, it is understood that a similar approach can also be used to treat other types of wounds using suitable autologous and/or allogeneic cells.

As disclosed herein, surprisingly small numbers of VSELs can be used to promote wound healing as effectively as large numbers. Moreover, small numbers of VSELs promote migration of cells from the wound site as effectively as large numbers. In view of the small small numbers of VSELs required and the presumed contribution to healing from cells of the wound site, in certain embodiments, effective compositions and treatments of the invention comprise allogeneic VSELs. Such allogeneic VSELs can be matched between donor and recipient, and the compositions may optionally comprise an immunosuppressant.

The compositions and methods of the invention disclosed herein are useful for treating a patient to ameliorate the formation of scars at a wound site. According to preferred embodiments, methods are provided to ameliorate, reduce, or decrease the formation of scars in a patient that has suffered a burn injury. According to preferred embodiments, methods are provided to treat, reduce the occurrence of, or reduce the probability of developing hypertrophic scars in a patient that has suffered an acute or chronic wound or injury.

The compositions and methods of the present invention may be used to treat chronic ischemic skin lesions. Accordingly to some embodiments, a composition comprising a suspension of stem/progenitor cells applied on the surface of and around chronic skin lesions, such as chronic ischemic skin lesions, chronic skin ulcers, and/or diabetic foot ulcers. Other types of chronic skin lesions include neuropathic and ischemic chronic cutaneous lesions (e.g., low-grade lesions, grade IV lesions, grade V lesions, etc.).

The sealants of the present invention may be administered to a subject using techniques well-known in the art, for example by injection or spray at the desired site, endoscopically, using a sponge-like carrier, pre-formed sealant or other methods known in the art. In one embodiment, the sealant is injected or sprayed and allowed to form a gel in situ.

In certain embodiments, the VSEL-containing sealants of the present invention may be delivered as a fine spray. For example, the sealants of the present invention are applied to wounds using a fibrin polymer spray system with a double-barreled syringe. For this, the fibrinogen component may be combined with the cellular component and this combination is applied as a using a fibrin polymer spray system with a double-barreled syringe that is capable of simultaneously applying the combined FC/cellular components and the thrombin component. The fibrinogen and thrombin polymerize to fibrin immediately and on contact with the wound bed. In other embodiments, the fibrin sealant (e.g., polymerized fibrin gel) may be applied topically to the site of the wound. For example, the fibrin sealant may be applied to topically to the site of the wound as a gel and spread to evenly cover the surface area of the wound (e.g., non-healing wound). As described herein, the stem cell-containing sealant may be covered with a suitable non-absorbent dressing, such as a spray on polymer which covers and maintains the stem cell containing sealant in position.

In certain embodiments, there is included a didactic component, which refers to an organized structure (e.g., polymer or scaffold) that provides a niche, habitat, or structural support that promotes propagation, migration, and/or differentiation of cells. According to some embodiments, the didactic component is a skin substitute or extracellular matrix (ECM) material. Skin substitutes include, but are not limited to, the following: acellular dressing (e.g., collagen bound to nylon fabric or mesh); autologous epidermal graft; acellular, allogeneic dermal graft; bovine collagen and chondroitin-6-sulfate; pig intestinal mucosa; human fibroblasts in an absorbable matrix; human fibroblasts and keratinocytes in a bovine collagen sponge; human fibroblasts and keratinocytes in a bovine collagen matrix. See e.g., U.S. Patent Publication No. 2007/0274963, incorporated herein by reference in its entirety.

According to some embodiments, the fibrin polymer spray system uses a very low CO₂ flow (i.e., less than about 10 p.s.i.) for fibrin delivery. Preferred ranges include from about 1 p.s.i. to about 10 p.s.i., from about 1 p.s.i. to about 9 p.s.i., from about 1 p.s.i. to about 8 p.s.i., from about 1 p.s.i. to about 7 p.s.i., from about 1 p.s.i. to about 6 p.s.i., from about 1 p.s.i. to about 5 p.s.i., from about 1 p.s.i. to about 4 p.s.i., from about 1 p.s.i. to about 3 p.s.i., from about 1 p.s.i. to about 2 p.s.i., from about 2 p.s.i. to about 5 p.s.i., from about 3 p.s.i. to about 5 p.s.i., or from about 4 p.s.i. to about 5 p.s.i. According to preferred embodiments, the fibrin polymer spray system uses a CO₂ flow at less than about 5 p.s.i. for fibrin delivery. According to preferred embodiments, the fibrin polymer spray system uses a CO₂ flow at less than about 4.5 p.s.i. for fibrin delivery. According to preferred embodiments, the fibrin polymer spray system uses a CO₂ flow at less than about 4 p.s.i. for fibrin delivery. According to preferred embodiments, the fibrin polymer spray system uses a CO₂ flow at less than about 3.5 p.s.i. for fibrin delivery. According to preferred embodiments, the fibrin polymer spray system uses a CO₂ flow at less than about 3 p.s.i. for fibrin delivery. According to preferred embodiments, the fibrin polymer spray system uses a CO₂ flow at less than about 2.5 p.s.i. for fibrin delivery. According to preferred embodiments, the fibrin polymer spray system uses a CO₂ flow at less than about 2 p.s.i. for fibrin delivery.

Both fibrinogen (containing the stem/progenitor cells) and thrombin may be diluted to optimally deliver a polymerized gel that immediately adheres to the wound, without run-off, and yet allows the stem/progenitor cells to remain viable and migrate from the gel.

The fibrin sealant of the present invention may be administered to the site of the wound as needed during the course of the healing process, such as once, twice, three times, four times, five times, ten times, twenty times, or more. According to preferred embodiments, the fibrin sealants are administered to the patient for at least two applications at least 1 week apart. According to preferred embodiments, the fibrin sealants are administered to the patient for at least two applications at least 2 weeks apart. According to preferred embodiments, the fibrin sealants are administered to the patient for at least three applications at least 1 week apart. According to preferred embodiments, the fibrin sealants are administered to the patient for at least three applications at least 2 weeks apart. According to preferred embodiments, the fibrin sealants are administered to the patient for at least four applications at least 1 week apart. According to preferred embodiments, the fibrin sealants are administered to the patient for at least four applications at least 2 weeks apart. According to preferred embodiments, the fibrin sealants are administered to the patient for at least five applications at least 1 week apart. According to preferred embodiments, the fibrin sealants are administered to the patient for at least five applications at least 2 weeks apart.

According to some embodiments, the present invention provides topical formulations that contain VSEL stem cells that are impregnated or seeded within a fibrin matrix. The VSEL stem cells used in the composition and methods of the present invention may be allogenic or autologous stem/progenitor cells. Potential sources of VSEL stem cells include, stem cells collected from umbilical cords, stem cells collected from peripheral blood, bone marrow-derived cells, stem cells mobilized from tissue, and stem cells that have been previously aliquoted and/or cyropreserved.

Collection of Stem Cells

The VSEL stem cells of the present invention may be collected from bone marrow, peripheral blood (preferably mobilized peripheral blood), spleen, cord blood, and combinations thereof. The VSEL stem cells may be collected from the respective sources using any means known in the art. Generally, the method of collecting VSEL stem cells from a subject will include collecting a population of total nucleated cells and further enriching the population for VSEL stem cells.

According to some embodiments, the VSEL stem cells of the present invention are collected from bone marrow. Bone marrow derived cells may be collected using any methods known in the art. According to some embodiments, bone marrow aspirates may be obtained from patients. According to some embodiments, single bone marrow aspirates may be obtained from patients. In some embodiments, a bone marrow aspirate contains a requisite number of VSELs and is incorporated directly into the wound repair composition of the invention.

According to preferred embodiments, the VSEL stem cells are collected from the peripheral blood of an individual. According to a preferred embodiment, the stem cells may be collected by an apheresis process, which typically utilizes an apheresis instrument. The apheresis instrument looks very much like a dialysis machine, but differs in that it is a centrifuge while a dialysis machine uses filtration technology. Stem cell collection can be accomplished in the privacy of the donors own home or in a collection center. Blood is drawn from one arm then enters the apheresis instrument where the stem cells are separated and collected. The rest of the whole blood is then returned to the donor. A registered nurse (RN) or other approved personnel places a needle into both arms of the subject in the same manner as a routine blood collection. The RN then operates the apheresis instrument that separates the blood elements (red cells, white cells, plasma) collecting the stem cells and returning the rest of the whole blood to the donor. The collection of stem cells requires approximately 2-4 hours during which the subject is at rest. Shortly after the apheresis collection, the bone marrow releases more stem cells into the blood stream to replace the harvested stem cells. The amount of stem cells collected is a very small fraction of a person's stem cells. In a healthy individual, the stem cells can rapidly multiply and replace the lost stem cells. Thus, the procedures of the invention do not deplete the body of stem cells. Many hundreds of thousands of apheresis collections take place each year for platelets, red cells, plasma and stem cells. It has been shown to be safe and effective technology.

According to some embodiments, stem cells are collected by the process of apheresis from adult or pediatric peripheral blood, processed to optimize the quantity and quality of the collected stem cells, optionally cryogenically preserved and used for autologous therapeutic purposes when needed after they have been thawed. According to a preferred embodiment, there is provided a method for collecting autologous adult stem cells from a human subject. The process may involve, for example, collecting stem cells from peripheral blood human subject using an apheresis process; at the time of collection, earmarking the collected cells for use by the human subject; and preserving the collected cells to maintain the cellular integrity of the cells. The human subject may be an adult human or non-neonate child. Accordingly, the above processes may further include the collection of adult or non-neonate child peripheral blood stem cells where the cells are then aliquoted into defined dosage fractions before cryopreservation so that cells can be withdrawn from storage without the necessity of thawing all of the collected cells.

WO/2011/069117 describes a method of isolation of stem cell populations from peripheral blood using sized-based separation. Fresh apheresed cells are lysed with 1× BD Pharm Lyse Buffer, in a ratio of approximately 1:10 (vol/vol) to remove red blood cells. After washing, cells are counted, and 2-2.5×10¹⁰ total nucleated cells are loaded onto the ELUTRA® Cell Separation System (CaridianBCT) at a concentration of 1×10⁸ cells/ml. Cells are then collected in 900 ml PBS+0.5% HSA media in each bag at different flow rates. Typically, six fractions are collected with a centrifugation speed of 2400 rpm. Finally, cells from all fractions are transferred into tubes and spun down at 600×g for 15 minutes. Size characteristics of the fractions are confirmed by evaluating SSC and FSC. As disclosed therein, Fraction 2 (50 mL/min) is highly enriched in VSELs and can be used to provide populations of VSELs for clinical applications. The procedure can be adapted to other equipment. The populations may be further purified by FACS.

hVSELs can be expanded through multiple passages. For example, by growing hVSELs on a feeder layer of OP9 cells or bone marrow-derived MSCs, the number of VSELs and VSEL-derived stem cells can be increased in a short period of time. Further, VSELs can be primed to differentiate towards hematopoietic/lymphopoietic lineage. For example, when cultured on a feeder layer of bone marrow-derived MSCs in serum-free medium in the presence of stem cell factor (SCF), FLT3 ligand (Flt3L), thrombopoietin (TPO) and basic Fibroblast Growth Factor (bFGF), a 200-fold expansion of hVSELs was achieved in a short time, allowing enough hVSELs to be generated from a patient to provide cells for transplants and booster transplants. Growing the VSELs in serum-free medium with SCF, TPO and Flt3-L also increases expression of the hematopoietic marker CD45. With time, hemangioblasts form which are progenitors of hematopoietic and endothelial cells (CD34⁺, CD133⁺, express Flk-1, mesodermal gene T (brachyury)). Hemangioblasts can be plated on methylcellulose to perform CFU-assays to test their hematopoietic potential. For example, colony forming unit-macrophage (CFU-M), colony forming unit-granulocyte (CFU-G) and Burst forming unit-erythrocyte (BFUE) can be observed.

Typically, collection may be performed on any person, including adult or a non-neonate child. Furthermore, collection may involve one or more collecting steps or collecting periods. For example, collection (e.g., using an apheresis process) may be performed at least two times, at least three times, or at least 5 times on a person. During each collecting step, the number of total nucleated cells collected per kilogram weight of the person may be one million (1×10⁶) or more (e.g., 1×10⁷, 1×10⁸, 1×10⁹, 1×10¹⁰, 1×10¹¹, 1×10¹², 1×10¹³, 1×10¹⁴, 1×10¹⁵). In preferred embodiments, the number of cells collected in a single collection session may be equal or greater than 1×10 ¹⁵ total nucleated cells, or at least on the order of 10¹⁴, or 10¹³, or 10¹², or 10¹¹, or 10¹⁰, or 10⁹, or 10⁸, or 10⁷, or 10⁶, or 10⁵ total nucleated cells, depending on the weight and age of the donor. The nucleated cells can be processed at the time of collection to enrich VSEL stem cells, which may optionally be aliquoted and/or cryopreserved. Alternatively, the nucleated cells may be aliquoted and/or cryopreserved, and enriched for VSEL stem cells at the time of use enrichment.

Depending on the situation and the quantity and quality of VSEL stem cells to be collected from the donor, it may be preferable to collect the VSEL stem cells from donors when they are at an “adult” or a “matured” age (the term “adult” as used herein refers to and includes adult and non-neonate, unless otherwise used in a particular context to take a different meaning) and/or at a certain minimum weight. For example, stem/progenitor cells are collected when the subject is within a range from 10 to 200 kg in accordance with one embodiment of the present invention, or any range within such range, such as 20 to 40 kg. In addition or in the alternative, it may be required that the subject be of a certain age, within a range from 2-80 years old (e.g., 2-10, 10-15, 12-18, 16-20, 20-26, 26-30, 30-35, 30-40, 40-45, 40-50, 55-60, 60-65, 60-70, and 70-80 years old) in accordance with one embodiment of the present invention.

Stem Cell Potentiating Agent

The amount of stem/progenitor cells circulating in the peripheral blood cell may be increased with the infusion of cell growth factors prior to collection, such as, for example, granulocyte colony stimulating factor (G-CSF). The infusion of growth factors is routinely given to bone marrow and peripheral blood donors and has not been associated with any long lasting untoward effects. Adverse side effects are not common but include the possibility of pain in the long bones, sternum, and pelvis, mild headache, mild nausea and a transient elevation in temperature. The growth factor is given 1-6 days before peripheral blood stem/progenitor cells are collected. 1-6 days after G-SCF is infused the peripheral blood stem/progenitor cells are sterilely collected by an apheresis instrument.

In a preferred embodiment, there is provided a method of mobilizing a significant number of peripheral blood stem/progenitor cells comprising the administration of a stem cell potentiating agent. The function of the stem cell potentiating agent is to increase the number or quality of the stem/progenitor cells that can be collected from the person. These agents include, but are not limited to, G-CSF, GM-CSF, dexamethazone, a CXCR4 receptors inhibitor, interleukin-1 (IL-1), interleukin-3 (IL-3), interleukin-8 (IL-8), PIXY-321 (GM-CSF/IL-3 fusion protein), macrophage inflammatory protein, stem cell factor, thrombopoietin and growth related oncogene, as single agents or in combination.

According to a preferred embodiment, the G-CSF is administered to a subject over a 1 to 6 day course, which ends upon apheresis of the subjects peripheral blood. Preferably, the G-CSF is administered to a subject at least twice over a 2 to 6 day period. For example, G-CSF may be administered on day 1 and day 3 or may be administered on day 1, day 3, and day 5 or, alternatively, day 1, day 2, and day 5. Most preferably, G-CSF is administered to a subject twice for consecutive days over a 3 day course. Thus, according to the preferred embodiment, G-CSF is administered to a subject on day 1 and day 2 followed by apheresis on day 3.

Additionally, according to preferred embodiments, a low dose G-CSF is administered to a subject. Thus, a subject may receive a dose of G-CSF of about 1 μg/kg/day to 8 μg/kg/day. Preferably, G-CSF is administered to a subject at a dose of about 2 to about 7 μg/kg/day or equivalent thereof. More preferably, G-CSF is administered to a subject at a dose of about 4 to about 6 μg/kg/day or equivalent thereof. For subcutaneous injections, the dose of G-CSF may be from about 50 μg to about 800 μg, preferably from about 100 μg to about 600 μg, more preferably from about 250 μg to 500 μg, and most preferably from about 300 μg to about 500 μg.

Accordingly to another preferred embodiment, antagonist or inhibitors of CXCR4 receptors may be used as a stem cell potentiating agents. Examples of CXCR4 inhibitors that have been found to increase the amount of stem/progenitor cells in the peripheral blood include, but are not limited to, AMD3100, ALX40-4C, T22, T134, T140 and TAK-779. See also, U.S. Pat. No. 7,169,750, incorporated herein by reference in its entirety. These stem cell potentiating agents may be administered to the person before the collecting step. For example, the potentiating agent may be administered at least one day, at least three days, or at least one week before the collecting step. Preferably, the CXCR4 inhibitors are administered to a subject at least twice over a 2 to 6 day period. For example, the CXCR4 inhibitors may be administered on day 1 and day 3 or may be administered on day 1, day 3, and day 5 or, alternatively, day 1, day 2, and day 5. Most preferably, the CXCR4 inhibitors are administered to a subject twice for consecutive days over a 3 day course. Thus, according to the preferred embodiment, the CXCR4 inhibitors are administered to a subject on day 1 and day 2 followed by apheresis on day 3.

The formulation and route of administration chosen will be tailored to the individual subject, the nature of the condition to be treated in the subject, and generally, the judgment of the attending practitioner. Suitable dosage ranges for CXCR4 inhibitors vary according to these considerations, but in general, the compounds are administered in the range of about 0.1 μg/kg to 5 mg/kg of body weight; preferably the range is about 1 μg/kg to 300 μg/kg of body weight; more preferably about 10 μg/kg to 100 μg/kg of body weight. For a typical 70 kg human subject, thus, the dosage range is from about 0.7 μg to 350 mg; preferably about 700 μg to 21 mg; most preferably about 700 μg to 7 mg. Dosages may be higher when the compounds are administered orally or transdermally as compared to, for example, i.v. administration.

Stem Cell Processing

In some embodiments of the invention, after collection, the stem/progenitor cells are processed according to methods known in the art (see, for example, Lasky, L. C. and Warkentin, P. I.; Marrow and Stem Cell Processing for Transplantation; American Association of Blood Banks (2002)). In an embodiment of the invention, processing may include the following steps: preparation of containers (e.g., tubes) and labels, sampling and/or testing of the collected material, centrifugation, transfer of material from collection containers to storage containers, the addition of cryoprotectant, etc. In some embodiments, after processing, some of the processed stem/progenitor cells can be made available for further testing.

The cells also may be processed, preferably before the preservation step is conducted. Processing may involve, for example, enrichment or depletion of cells with certain cell surface markers. Any cell surface marker, including the cell surface markers listed anywhere in this specification may be used as a criteria for enrichment or depletion. Furthermore, processing may involve analyzing at least one characteristic of one cell in the one population of stem/progenitor cells or the at least one population of non-stem/progenitor cells. The characteristic may be a DNA or RNA sequence. For example, the genomic DNA or RNA may be partially or completely sequenced (determined). Alternatively, specific regions of the DNA or RNA of a cell may be sequenced. For example, nucleic acids from a cell or a cell population may be extracted. Specific regions of these nucleic acid may be amplified using amplification probes in an amplification process. The amplification process may be, for example PCR or LCR. After amplification, the amplimers (products of amplification) may be sequenced. Furthermore, the DNA and RNA may be analyzed using gene chips, using hybridization or other technologies.

Tissue typing of specific kinds may be used for sample identification or for the use of these VSEL stem cells for possible allogeneic use. This type of information may include genotypic or phenotypic information. Phenotypic information may include any observable or measurable characteristic, either at a macroscopic or system level or microscopic, cellular or molecular level. Genotypic information may refer to a specific genetic composition of a specific individual organism, including one or more variations or mutations in the genetic composition of the individual's genome and the possible relationship of that genetic composition to disease. An example of this genotypic information is the genetic “fingerprint” and the Human Leukocyte Antigen (HLA) type of the donor. In some embodiments of the invention the stem/progenitor cells will be processed in such a way that defined dosages may be identified and aliquoted into appropriate containers.

Stem Cell Enrichment or Sorting

The enrichment procedure preferably includes sorting the cells by size and/or cellular markers. The pattern of markers express by stem cells may also be used to sort and categorize stem cells with greater accuracy. Any means of characterizing, including the detection of markers or array of markers, may be used to characterized and/or identify the cells obtained through the embodiments disclosed herein. For example, as provided above, VSEL stem cells are known to express certain patterns of markers, and the cells collected by the processes described herein may be sorted on the basis of these known patterns.

In Vitro Propagation of Stem Cells

VSEL stem cells must be present in sufficient numbers for meaningful topical application. In certain embodiments, VSEL stem cells are cultured and expanded. Proper characterization of the cultured cells is also important to ensure a therapeutically effective amount. According to preferred embodiments, the number of VSEL stem cells collected during the collection process is sufficient for the therapeutic application (e.g., wound healing). That is, in vitro propagation of the collected cells is not necessary. In other embodiments, it may be desirable to propagate VSEL stem cells, for example prior to or following cryopreservation. For example, in certain instances, subjects may already have stored (e.g., cryopreserved) their own VSEL stem cells. In other instances, it may be desirable to treat a subject employing stored/preserved allogenic VSELs of another donor.

The propagation of the stem cells may be achieved using any known method. The stem cells of the present invention may be propagated on: 1) a tissue culture substrate in a stem cell medium that favors the maintenance of stem cells in a undifferentiated or dedifferentiated condition; 2) on fibroblast feeder layers that support cell growth and proliferation and inhibition of differentiation; or 3) a combination of both 1 and 2. In a preferred embodiment, the tissue culture substrate is coated with an adhesive or other compound or substance that enhances cell adhesion the substrate (e.g., collagen, gelatin, or poly-lysine, etc.). Collagen-coated plates are most preferred. Where fibroblast feeder cells are utilized, mouse or human fibroblasts are preferably used; alone or in combination. It is preferred that the feeder cells are treated to arrest their growth, which may be accomplished by irradiation or by treatment with chemicals such as mitomycin C that arrests their growth. Most preferably, the fibroblast feeder cells are treated with mitomycin C. In preferred embodiments, the fibroblast feeder layer has a density of approximately 25,000 human and 70,000 mouse cells per cm², or 75,000 to 100,000 mouse cells per cm². Preferably, the stem cells are cultured for a period of 4 to 24 days, and preferably for a period of 7 to 14 days. Preferably, the stem cells are grown on a fibroblast feeder layer, such as mitomycin treated MEF cells, for a period of about 4 to 14 days, and preferably from 7 to 10 days.

Kits

Kits are also contemplated within the scope of the invention. A typical kit can comprise a fibrin sealant comprising an FC and a thrombin component. In one embodiment, the kit further comprises stem/progenitor cells or an admixture of stem/progenitor cells for incorporation into the fibrin sealant. In one aspect, each component may be included in its own separate storage container, vial or vessel. In a related aspect, the stem/progenitor may be in admixture with the FC component, and the thrombin component may be in a separate storage container. In a related aspect, the stem/progenitor may be in admixture with the thrombin component, and the FC component may be in a separate storage container. In a related embodiment, the storage container is a vial, a bottle, a bag, a reservoir, tube, blister, pouch, patch or the like. One or more of the constituents of the formulation may be lyophilized, freeze-dried, spray freeze-dried, or in any other reconstitutable form. Various reconstitution media can further be provided if desired.

The components of the kit may be in either frozen, liquid or lyophilized form. It is further contemplated that the kit contains suitable devices for administering the fibrin gel to a subject. In a further embodiment, the kit also contains instructions for preparing and administering the fibrin sealant.

According to some embodiments, the fibrin sealants of the present invention, and kits thereof, may further comprise growth factors known to be involved in the healing process. Such growth factors include, but are not limited to platelet-derived growth factor-BB (PDGF-BB), transforming growth factor-β1 (TGF-β1), and platelet derived growth factor (PDGF). The growth factors may be added in any concentration that provides an adequate delayed release formulation, within a range of 1 ng/ml to 1 mg/mL of the growth factors. Exemplary concentrations of growth factor in the fibrin sealant include, but are not limited to 1 ng/ml, 5 ng/ml, 10 ng/ml, 15 ng/ml, 20 ng/ml, 40 ng/ml, 50 ng/ml, 100 ng/ml, 250 ng/ml, 500 ng/ml, 1 μg/ml, 5 μg/ml, 10 μg/ml, 25 μg/ml, 50 μg/ml, 100 μg/ml, 250 μg/ml, 500 μg/ml, 750 μg/ml and 1 mg/ml. See e.g., U.S. Patent Publication Nos. 2009/0075881 and 2008/0181879, incorporated herein by reference in their entireties.

Definitions

As used herein the terms “fibrin sealant,” “fibrin gel,” “fibrin adhesive,” “fibrin clot” or “fibrin matrix” are used interchangeably and refer to a three-dimensional network comprising at least a fibrinogen complex (FC) component and a thrombin component, which can act as a scaffold for cell growth and release of a bioactive materials over time.

The term “calcic thrombin” as used herein includes thrombin in the presence of calcium.

As used herein, “stem cells” refer to cells that can give rise to one or more cell lineages. Included are progenitor cells, totipotent cells, pluripotent cells, embryonic cells or post natal and adult cells. Also included are tissue-specific cells, including, but not limited to, cells committed to a particular lineage capable of undergoing terminal differentiation, cells that derive from tissue resident cells, and circulating cells that have homed to specific tissues.

The term “therapeutically effective amount” is used to denote treatments at dosages effective to achieve the therapeutic result sought. Furthermore, one of skill will appreciate that the therapeutically effective amount of the composition of the invention may be lowered or increased by fine tuning and/or by administering more than one composition of the invention (e.g., by the concomitant administration of different populations of cells such as genetically modified cells or type of stem/progenitor cells), or by administering a composition of the invention with another compound to enhance the therapeutic effect (e.g., synergistically). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect. The methods of the invention can thus be used, alone or in combination with other well known wound healing therapies, to treat a patient having acute or chronic wounds. One skilled in the art will readily understand advantageous uses of the invention, for example, by reducing healing time and outcome for a patient suffering from acute or chronic wounds.

Technical and scientific terms used herein have the meaning commonly understood by one of skill in the art to which the present invention pertains, unless otherwise defined.

The methods of the present invention are intended for use with any subject that may experience the benefits of the methods of the invention. Thus, in accordance with the invention, “subjects”, “patients” as well as “individuals” (used interchangeably) include humans as well as non-human subjects, particularly domesticated animals.

As used herein, an “allogeneic cell” refers to a cell that is not derived from the individual to which the cell is to be administered, that is, has a different genetic constitution than the individual. An allogeneic cell is generally obtained from the same species as the individual to which the cell is to be administered. For example, the allogeneic cell can be a human cell, as disclosed herein, for administering to a human patient such as a cancer patient.

As used herein, a “genetically modified cell” refers to a cell that has been genetically modified to express an exogenous nucleic acid, for example, by transfection or transduction.

It is understood that modifications which do not substantially affect the activity of the various embodiments of this invention are also provided within the definition of the invention provided herein. Accordingly, the following examples are intended to illustrate but not limit the present invention. While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

### EXAMPLES

The following examples describe the cell culture and characterization, delivery system, and successful use of topically applied VSEL stem cells to accelerate the healing of human and experimental murine wounds.

### Example 1

Collection of VSELs—Human VSELs are isolated from G-CSF-mobilized peripheral blood by leukapheresis followed by a 3-step purification procedure. First, smaller cells are enriched by counterflow centrifugal elutriation using an Elutra cell separator (Terumo BCT). Elutra fractions enriched in VSELs (defined for the purposes of purification as Lin⁻/CD45⁻/CD34⁺ and/or Lin⁻/CD45⁻/CD133⁺ cells) are then positively selected using anti-CD34 and anti-CD133 microbeads, and then purified to >98% Lin− CD45− CD34/133+ objects by flow cytometric sorting.

### Example 2

Fibrin Spray System to Deliver Cells Topically to Wounds—The fibrin delivery method made use of the TISSEEL VH® fibrin sealant system and the TISSOMAT® application device and spray set (Baxter Healthcare, Glendale, Calif.). This preparation contains human fibrinogen and thrombin. The following protocol was used to make a fibrin gel with final concentrations of 5 mg/mL fibrinogen and 25 U/mL thrombin, utilizing a 1 mL TISSEEL VH® kit (Baxter). This kit utilizes two liquid phases that can be either extruded through a dual chamber applicator or sprayed through the applicator with an inert gas carrier. To make the thrombin component, 1 mL of calcium chloride (CaCl₂) mixture from the kit was added using a sterile syringe to the 1 mL bottle of thrombin, and the mixture was allowed to dissolve. One part of this solution was then added to 9 parts of sterile 30 mM CaCl₂ in normal saline (0.9% sodium chloride [NaCl]). The fibrinogen/sealer protein component was made by adding 1 mL of sterile normal saline to the sealer protein bottle. One part of this solution was then added to 9 parts of sterile normal saline. The protease inhibitor aprotinin included with the kit was not used. All solutions were used within 4 hours. The total volume of fibrin gel was predetermined by the size of the wound to be covered. At the time of application to each wound, a small amount of the fibrin gel was placed on a tissue culture plate, covered in media, and incubated under standard conditions to verify and confirm cell viability and migration of the cells from the fibrin.

### Example 3

VSEL stem cells are applied up to four times to wounds using a fibrin polymer spray system with a double-barreled syringe. Both fibrinogen (containing the VSELs) and thrombin are diluted to optimally deliver a polymerized gel that immediately adheres to the wound, without run-off, and yet allows the VSELs to remain viable and migrate from the gel. Sequential adjacent sections from biopsy specimens of the wound bed after VSEL application show cells immunostained for VSEL markers. Generation of new elastic fibers can be observed using stains and antibodies to human elastin.

### Example 4

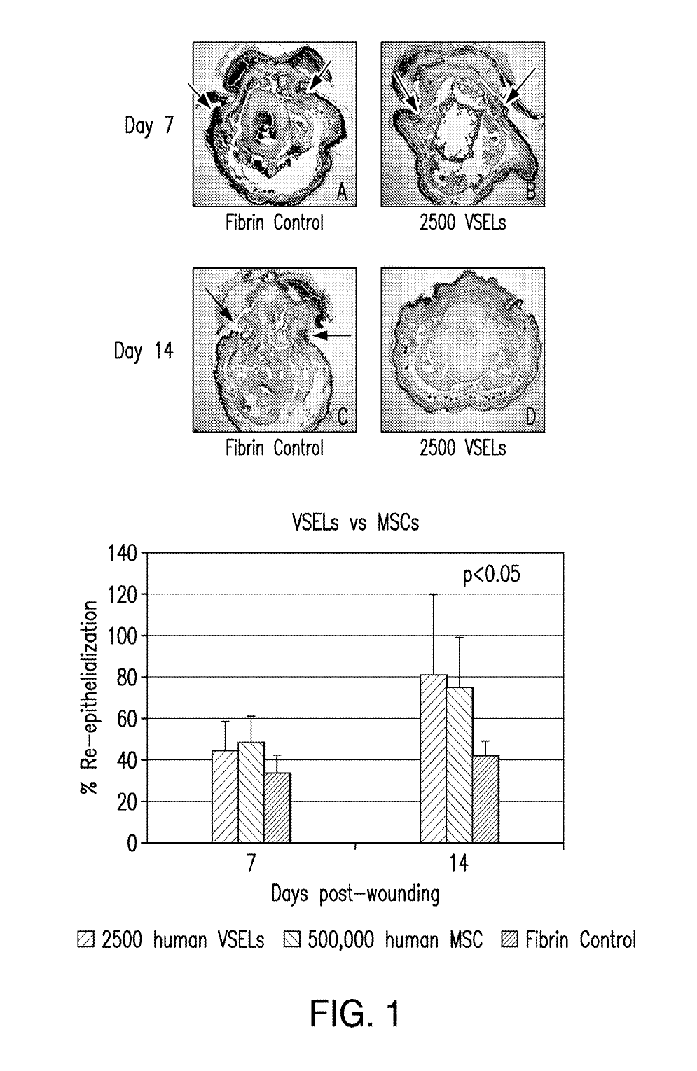

Human VSELs Accelerate Healing in a SCID Mouse Tail Wound Model. The capacity of human VSELs to promote tail wound healing was compared with MCSs. Human VSELs and MSCs were administered in a fibrinogen-containing sealant according to the invention. The dosage of MSCs was 200× the dosage of VSELs (500,000 MSCs compared to 2,500 VSELs). Tail wound healing was evaluated by reepithelialization. Seven days after wounding and treatment, increased reepithelialization was observed using both cell types compared to fibrin control. Only a small amount of reepithelialization was observed under control conditions. At 14 days following wounding and treatment, reepithelialization continued for both the MSC group and VSEL group, with little further improvement in the fibrin control group. Overall, the same amount of reepithelialization was observed for the VSEL group (2,500 cells) as for the MSC group (500,000 cells), which as about twice as much as was observed for the fibrinogen control. (FIG. 1)

### Example 5

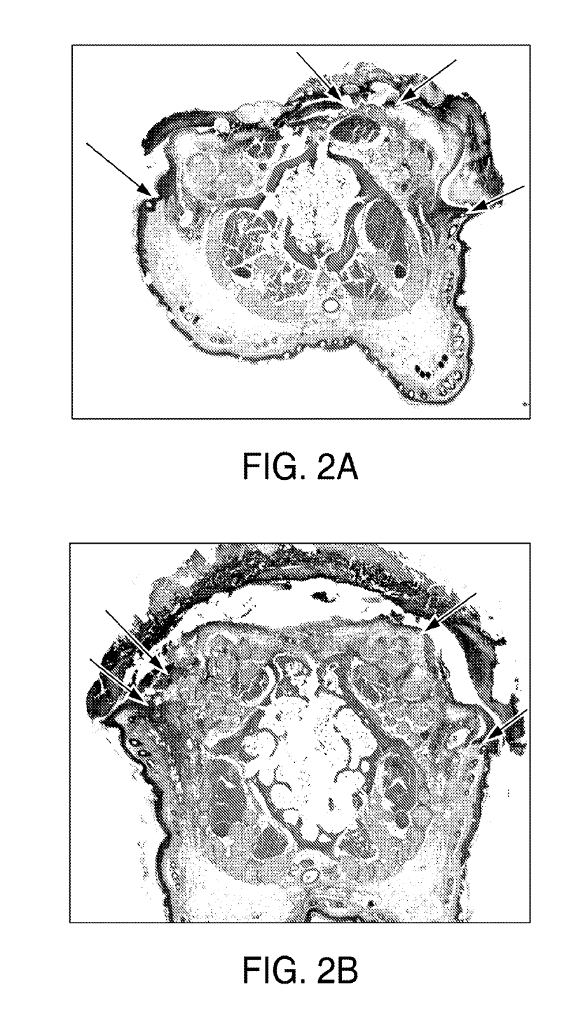

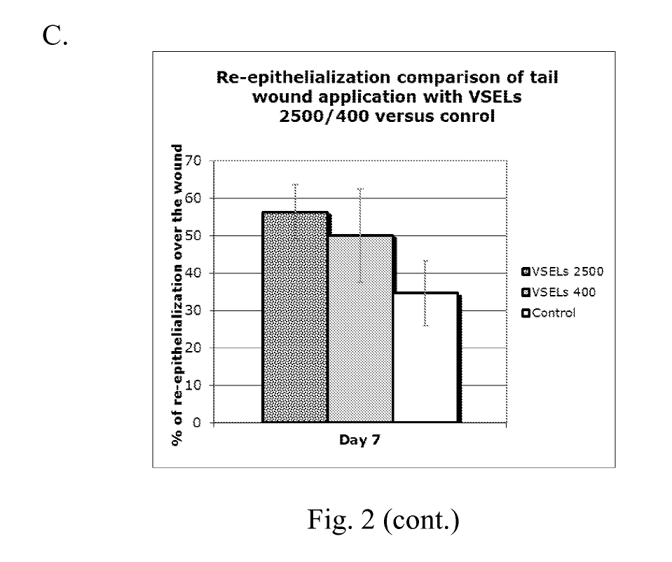

Human VSELs Accelerate Healing in a SCID Mouse Tail Wound Model. Two doses of human VSELs were tested to investigate dependence of wound healing on the number of VSELs administered. VSELs administered in two amounts (2,500 or 400 VSELs) were compared to fibrinogen control. Seven days after wounding and treatment, increased reepithelialization was observed using both VSEL amounts (FIG. 2A) compared to fibrin control (FIG. 2B). % reepithelialization attained by the two doses, compared to fibrin control is depicted in FIG. 2C.

### Example 6

Treatment of Secondary or Full-Thickness Burns—Following debridement of dead and damaged tissue, a VSEL-containing composition of the invention is applied to the wound. A interim cover is applied, for example, cadaver skin (allograft), pig skin (xenograft), or other protective layer. The resulting VSEL-containing layer promotes wound bed preparation on its own and in conjunction with the selected interim covering, enhancing wound bed preparation by increasing vascularity in the wound bed, including promoting angiogenesis with enhanced capillary ingrowth. A VSEL-constaining composition of the invention can also be applied in conjunction with the subsequent autograft, as well as at the sites from which autografts are taken.

We claim:

1. A composition for treating a wound, comprising a matrix and a cellular component;
wherein the cellular component comprises an amount of VSEL stem cells for application to the wound in a dosage of 10⁵ or fewer VSEL stem cells/cm².

2. The composition of claim 1, wherein the matrix comprises a fibrinogen complex (FC) component and a thrombin component.

3. The composition of claim 1, wherein the VSEL stem cells are obtained from bone marrow, peripheral blood, cord blood, or a combination thereof.

4. The composition of claim 1, wherein the VSEL stem cells are lin⁻/CD45⁻/CD34⁺, lin⁻/CD45⁻/CD133⁺, lin⁻/CD45⁻/CXCR4⁺, or lin⁻/CD45⁻/CXCR4⁺/CD133⁺/CD34⁺.

5. The composition of claim 1, wherein the VSEL stem cells are lin⁻/CD45⁻/Sca-1⁺ or lin⁻/CD45⁻/CD⁺.

6. A method of treating a cutaneous wound, comprising:
a) combining VSEL stem cells with a fibrin sealant to form a wound sealant; wherein the fibrin sealant comprises calcic thrombin and fibrinogen, wherein the amount of VSELs in the wound sealant provides a dosage of 10⁵ or fewer VSEL stem cells/cm² when administered to the cutaneous wound, and
b) administering the fibrin sealant to the cutaneous wound.

7. The method of claim 6, which comprises administering the wound sealant at least two times over the span of three weeks.

8. The method of claim 6, wherein the wound sealant is topically applied to the site of the wound.

9. The method of claim 6, wherein the wound sealant is administered to the site of the wound in the form of a spray at a CO₂ psi of less than 5 psi.

10. The method of claim 6, which comprises applying a skin substitute at the site of the wound.

11. The method of claim 6, wherein the wound is the result of a burn.

12. The method of claim 6, wherein the wound is a scleroderma ulcer.

13. A method of ameliorating the formation of scars at a wound site, comprising:
a) combining VSEL stem cells with a fibrin sealant to form a wound sealant, said fibrin sealant comprising calcic thrombin and fibrinogen, wherein the concentration of calcic thrombin is about 25 U/ml, wherein the concentration of fibrinogen is from about 2 to about 5 mg/ml, and wherein the final concentration of VSEL stem cells provides a dosage to the wound of 10⁵ or fewer VSEL stem cells/cm², and
b) administering the wound sealant to a wound site, wherein the wound sealant is administered in the form of a polymerized gel or spray.

14. The method of claim 13, which comprises administering the wound sealant at least two times over the span of three weeks.

15. The method of claim 13, wherein the wound sealant is administered to the site of the wound in the form of a spray at a CO₂ psi of less than 5 psi.

16. The method of claim 13, wherein the fibrin sealant is topically applied to the site of the wound.

17. The method of claim 13, wherein a skin substitute is applied to the site of the wound.

18. The method of claim 13, wherein the wound is the result of a burn.

19. The method of claim 13, wherein the wound is a scleroderma ulcer.

20. The method of claim 13, wherein the scar is a hypertrophic scar.

21. A method of making a wound sealant comprising;
providing a fibrinogen complex (FC) component, a calcic thrombin component, and a cellular component;
adding the cellular component to the FC component before admixture of the FC component with the calcic thrombin component; and
adding the calcic thrombin component to the combined FC/cellular component mixture, wherein the concentration of fibrinogen is from about 2 to about 5 mg/ml, and wherein the final concentration of stem/progenitor cells provides a dosage of 10⁵ or fewer VSEL stem cells/cm² when administered to the wound.

22. A kit for preparing a wound sealant comprising, a) a first vial or first storage container containing a fibrinogen complex component and a VSEL stem cell component, wherein the concentration of fibrinogen is from about 2 to about 5 mg/ml, wherein the VSEL stem cell component is in an amount suitable to produce a dosage of 10⁵ or fewer VSEL stem cells/cm² when administered to a wound, and b) a second vial or second storage container having a thrombin component, said kit further containing instructions for use thereof.

23. A kit for preparing a wound sealant comprising, a) a first vial or first storage container containing a fibrinogen complex component, wherein the concentration of fibrinogen is from about 2 to about 5 mg/ml, b) a second vial or second storage container having a thrombin component, and c) a third vial or third storage container having a VSEL stem cell component in an amount suitable to produce a dosage of 10⁵ or fewer VSEL stem cells/cm² when administered to a wound, said kit further containing instructions for use thereof.
